(19) United States
(12) Patent Application Publication
(10) Pub. No.: US 2024/0182968 A1
(43) Pub. Date: Jun. 6, 2024

(54) INCREASING CAPTURE EFFICIENCY OF SPATIAL ASSAYS

(71) Applicant: 10x Genomics, Inc., Pleasanton, CA (US)

(72) Inventor: Felice Alessio Bava, Pleasanton, CA (US)

(21) Appl. No.: 18/438,896

(22) Filed: Feb. 12, 2024

Related U.S. Application Data
(62) Division of application No. 17/165,342, filed on Feb. 2, 2021, now Pat. No. 11,898,205.
(60) Provisional application No. 62/969,469, filed on Feb. 3, 2020.

(51) Int. Cl. C12Q 1/6876; C12Q 1/6837

(52) U.S. Cl. CPC C12Q 1/6876; C12Q 1/6837; C12Q 2521/507; C12Q 2525/173; C12Q 2531/125; C12Y 605/01001; C12Y 605/01003

(57) ABSTRACT

This disclosure relates to methods for increasing capture efficiency of a spatial array using rolling circle amplification of a padlock probe that hybridizes to a capture probe. Also provided are methods for using such spatial arrays to detect a biological analyte in a biological sample.

### CROSS-REFERENCE TO RELATED APPLICATION

This application is a divisional application of U.S. patent application Ser. No. 17/165,342, filed Feb. 2, 2021, which claims priority to U.S. Provisional Patent Application No. 62/969,469, filed Feb. 3, 2020. The contents of these applications are incorporated herein by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Genetic material, and related gene and protein expression, influences cellular fate and behavior. The spatial heterogeneity in developing systems has typically been studied via RNA hybridization, immunohistochemistry, fluorescent reporters, or purification or induction of pre-defined subpopulations and subsequent genomic profiling (e.g., RNA-seq). Such approaches, however, rely on a small set of pre-defined markers, therefore introducing selection bias that limits discovery and making it costly and laborious to localize RNA transcriptome-wide.

### SUMMARY

This disclosure relates to methods of increasing capture efficiency of a spatial array.

In one aspect, disclosed herein are methods for preparing a spatial array, the method comprising: (a) contacting a substrate comprising a plurality of capture probes with a padlock probe, wherein (i) a capture probe of the plurality of capture probes comprises a first docking sequence, a spatial barcode, a capture domain, and a second docking sequence; and (ii) the padlock probe comprises: a first docking padlock sequence at its 3′ end that is complementary to the first docking sequence of the capture probe, and a second docking padlock sequence at its 5′ end that is complementary to the second docking sequence of the capture probe; (b) hybridizing the first docking sequence to the first docking padlock sequence and hybridizing the second docking sequence to the second docking padlock sequence; (c) ligating the padlock probe, thereby generating a ligated padlock probe that comprises a capture domain; and (d) amplifying the ligated padlock probe, thereby generating a padlock probe sequence with multiple copies of the capture domain. In another aspect, provided herein are methods of increasing capture efficiency of a spatial array comprising: (a) contacting a substrate comprising a plurality of capture probes with a first oligonucleotide; wherein (i) a capture probe of the plurality comprises a first docking sequence, a second docking sequence, a barcode, and a capture domain; and (ii) the first oligonucleotide comprises a first complementary sequence complementary to the first docking sequence of the capture probe and a second complementary sequence complementary to the second docking sequence of the capture probe; (b) hybridizing the first docking sequence of the capture probe to the first complementary sequence of the first oligonucleotide and hybridizing the second docking sequence of the capture probe to the second complementary sequence of the first oligonucleotide; (c) ligating the 3′ end of the first oligonucleotide to the 5′ end of the first oligonucleotide, thereby forming a ligation product; and (d) amplifying the ligation product using rolling circle amplification.

Also provided herein are methods of increasing capture efficiency of a spatial array comprising: (a) contacting a substrate comprising a plurality of capture probes with a first oligonucleotide and a second oligonucleotide; wherein (i) a capture probe of the plurality comprises a first docking sequence, a barcode, and a capture domain; (ii) a second oligonucleotide comprises a second docking sequence; (iii) the first oligonucleotide comprises a first complementary sequence that is complementary to the first docking sequence of the capture probe and a second complementary sequence that is complementary to the second docking sequence of the second oligonucleotide; (b) hybridizing the first docking sequence of the capture probe to the first complementary docking sequence of the first oligonucleotide and hybridizing the second docking sequence of the second oligonucleotide to the second complementary docking sequence of the first oligonucleotide; (c) ligating the 3′ end of the first oligonucleotide to the 5′ end of the first oligonucleotide, thereby forming a ligation product; and (d) amplifying the ligation product using rolling circle amplification to form an amplified ligation product.

Also provide herein are methods of increasing capture efficiency of a spatial array comprising: (a) contacting a substrate comprising a plurality of capture probes with a first oligonucleotide; wherein (i) a capture probe of the plurality comprises a first docking sequence, a second docking sequence, a barcode, and a capture domain; (ii) the first oligonucleotide comprises a first complementary sequence complementary to the first docking sequence of the capture probe and a second complementary sequence complementary to the first docking sequence of the capture probe; (b) hybridizing the first docking sequence and the second docking sequence of the capture probe to the first complementary docking sequence and the second complementary docking sequence of the oligonucleotide; (c) extending the 3′ end of the first oligonucleotide to form an extended 3′ end of the first oligonucleotide; (d) ligating the extended 3′ end to the 5′ end of the first oligonucleotide to form a ligation product; and (e) amplifying the ligation product using rolling circle amplification to form an amplified ligation product.

In some instances, the spatial barcode and the capture domain are located between the first docking sequence and the second docking sequence of the capture probe. In some instances, the ligated padlock probe further comprises a primer sequence, or a complement thereof, and a spatial domain, or a complement thereof. In some instances, the amplifying comprises rolling circle amplification. In some instances, the padlock probe comprises a 3′OH group, wherein the 3′OH group is a primer for the rolling circle amplification.

In some instances, the step of hybridizing the padlock probe to the capture probe utilizes a splint oligonucleotide that comprises a sequence complementary to the capture probe and a sequence complementary to the padlock probe.

In some instances, the ligating step further includes extending the padlock probe via a nucleic acid extension reaction using the capture probe as a template. In some instances, the extending occurs prior to the ligating step.

In some instances, the capture domain comprises a poly(T) sequence, a random sequence, a semi-random sequence or a fixed sequence.

In some instances, the ligation step comprises using enzymatic ligation or chemical ligation. In some instances, the enzymatic ligation utilizes a ligase, wherein the ligase is one or more of a T4 RNA ligase (Rnl2), a splintR ligase, a single stranded DNA ligase, or a T4 DNA ligase.

In some embodiments, a portion of the first oligonucleotide is at least 80%, at least 85%, at least 90%, at least 95%, at least 96%, at least 97%, at least 98% or at least 99% complementary to the capture probe. In some embodiments, a portion of the first oligonucleotide is fully complementary to the capture probe. In some embodiments, a portion of the second oligonucleotide is at least 80%, at least 85%, at least 90%, at least 95%, at least 96%, at least 97%, at least 98% or at least 99% complementary to the capture probe. In some embodiments, a portion of the second oligonucleotide is fully complementary to the capture probe.

In some embodiments, a method described herein further comprises copying the capture domain sequence using a DNA polymerase.

In some embodiments, a product of the rolling circle amplification comprises amplifying multiple copies of a capture sequence and multiple copies of the spatial barcode.

In some embodiments, the capture sequence is an oligo d(T) sequence. In some embodiments, the first oligonucleotide comprises a functional sequence. In some embodiments, the functional sequence is a primer sequence. In some embodiments, the first oligonucleotide comprises 3′OH group, wherein the 3′OH group is a primer for the rolling circle amplification.

In some embodiments, the second oligonucleotide comprises a functional sequence. In some embodiments, the functional sequence is a primer sequence. In some embodiments, the second oligonucleotide comprises 3′OH group, wherein the 3′OH group is a primer for the rolling circle amplification.

In some embodiments, the ligation step comprises using enzymatic ligation or chemical ligation. In some embodiments, the enzymatic ligation utilizes a ligase. In some embodiments, the ligase is one or more of a T4 RNA ligase (Rnl2), a splintR ligase, a single stranded DNA ligase, or a T4 DNA ligase.

In some embodiments, the amplifying is isothermal. In some embodiments, the amplifying is not isothermal.

In some embodiments, the rolling circle amplification comprises using a Phi29 polymerase.

In some embodiments, a method described herein further comprises a step of washing away any oligonucleotides that do not hybridize after the hybridizing step.

In some embodiments, a method described herein further comprises spatially profiling an analyte in a biological sample.

In some embodiments, spatially profiling an analyte in a biological sample comprises: (a) contacting the biological sample with the substrate comprising the amplified ligation product; (b) releasing the analyte from the biological sample, wherein the analyte is bound by the amplified ligation product at a distinct spatial position of the substrate; (c) detecting the biological analyte bound by the amplified ligation product; and (d) correlating the biological analyte with the barcode at the distinct spatial position of the substrate.

In some instances, the methods disclosed herein further include spatially profiling an analyte in a biological sample. In some instances, spatially profiling the analyte includes the steps of: contacting the spatial array with the biological sample; hybridizing the analyte or analyte derivative to a capture domain of the multiple copies of the capture domain; and determining (i) all or a part of the sequence of the analyte or analyte derivative, or a complement thereof, and (ii) all or a part of the sequence of the spatial barcode, or a complement thereof, and using the determined sequence of (i) and (ii) to determine the abundance and the location of the analyte in the biological sample.

In some instances, the methods disclosed herein further include contacting the biological sample with a permeabilization agent, wherein the permeabilization agent is selected from an organic solvent, a detergent, and an enzyme, or a combination thereof.

In some instances, the determining step comprises sequencing.

In some instances, the methods disclosed herein further include imaging the biological sample. In some embodiments, the biological sample is a tissue section. In some embodiments, the biological sample is a formalin-fixed, paraffin-embedded (FFPE) sample, a frozen sample, or a fresh sample. In some embodiments, the biological sample is an FFPE sample.

In some embodiments, the analyte is a DNA molecule or an RNA molecule. In some embodiments, the RNA molecule is mRNA. In some instances, the analyte is an RNA molecule or a protein, or both.

Also disclosed herein are kits. In some instances, a kit as disclosed herein includes (a) an array comprising a plurality of primers; (b) a plurality of padlock probes; (c) a plurality of enzymes comprising a polymerase and a ligase; and (d) instructions for performing any of the methods disclosed herein.

Also disclosed herein are compositions. In some instances, a composition disclosed herein includes (a) a substrate comprising a plurality of capture probes, wherein a capture probe of the plurality of capture probes comprises a first docking sequence, a spatial barcode, a capture domain, and a second docking sequence; (b) a plurality of amplified padlock probes, wherein an amplified padlock probe of the plurality of amplified padlock probes comprises: (i) a first docking padlock sequence that is complementary to the first docking sequence of the capture probe, (ii) a second docking padlock sequence that is complementary to the second docking sequence of the capture probe, and (iii) a sequence complementary to the capture domain; and wherein the amplified padlock probe is hybridized to the capture probe. In some instances, an analyte hybridized to the amplified padlock probe in the composition. In some instances, the amplified padlock probe further comprises a primer sequence, or a complement thereof, and a spatial domain, or a complement thereof.

All publications, patents, patent applications, and information available on the internet and mentioned in this specification are herein incorporated by reference to the same extent as if cach individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the sample.

FIG. 3 is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A-6C are schematics illustrating how streptavidin cell tags can be utilized in an array-based system to produce a spatially-barcoded cells or cellular contents.

FIG. 7 is a schematic illustrating the generation of a probe using a padlock probe hybridized to a capture probe (top) and a padlock probe, a capture probe, and an oligonucleotide.

FIG. 8 is a schematic showing a padlock probe.

FIG. 9 is a schematic showing the annealing of multiple RNAs to a probe.

### DETAILED DESCRIPTION

### I. Introduction

Spatial analysis methods using capture probes and/or analyte capture agents provide information regarding the abundance and location of an analyte (e.g., a nucleic acid or protein). The efficiency of spatial analysis using arrays with capture probes depends, at least in part, on the density of the probes on the array or the density of the analytes captured on the array. That is, on how many capture probes can be printed on the surface of a slide or how many RNA molecules can be captured. Disclosed herein are methods and compositions for increasing the efficiency of spatial analysis by increasing the number of interactions between the capture probe and the analyte. In this way, analyte detection signal is increased, thus increasing the capturing efficiency, sensitivity, and the resolution of detection on the spatial array.

Traditionally, these methods identify a singular molecule at a location. Extending these methods to study interactions between two or more analytes would provide information on the interactions between two or more analytes at a location in a biological sample. Analyte capture agents as provided herein comprises an analyte binding moiety affixed to an oligonucleotide. The oligonucleotide comprises a sequence that uniquely identifies the analyte and moiety. Further, nearby oligonucleotides affixed to a different moiety in a nearby location can be ligated to the first oligonucleotide and then can be detected using the spatial methods described herein. The methods disclosed herein thus provide the ability to study the interaction between two or more analytes in a biological sample.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434): 1463-1467, 2019; Lee et al., Nat. Protoc. 10(3):442-458, 2015; Trejo et al., PLOS ONE 14(2):c0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

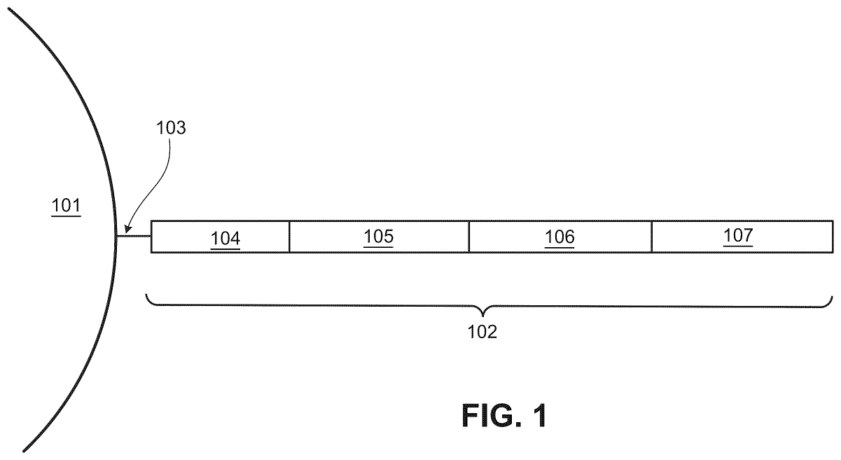

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

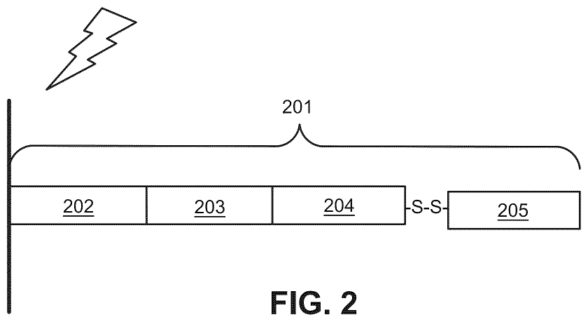

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

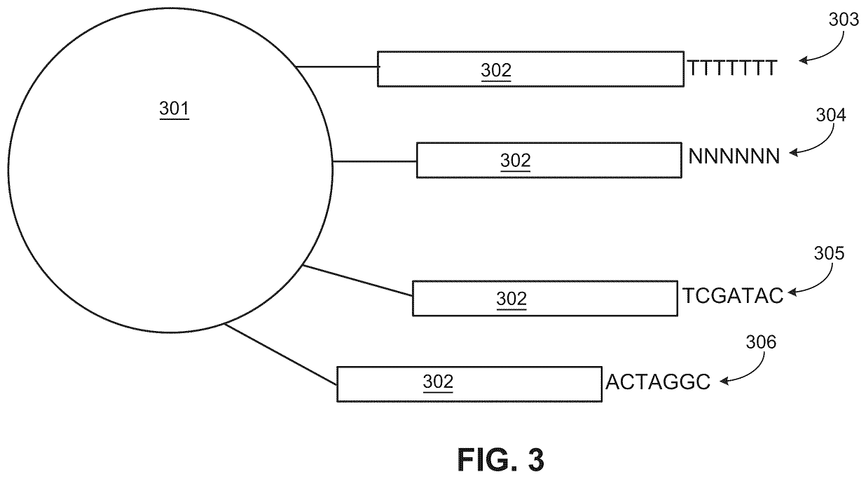

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. Sec, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

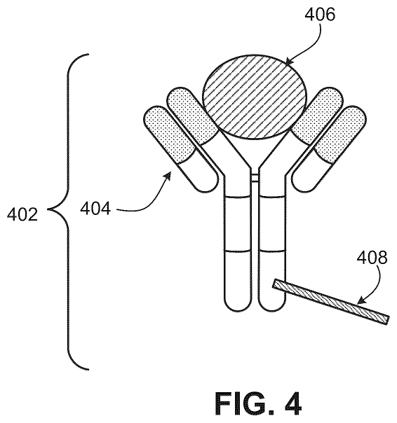

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

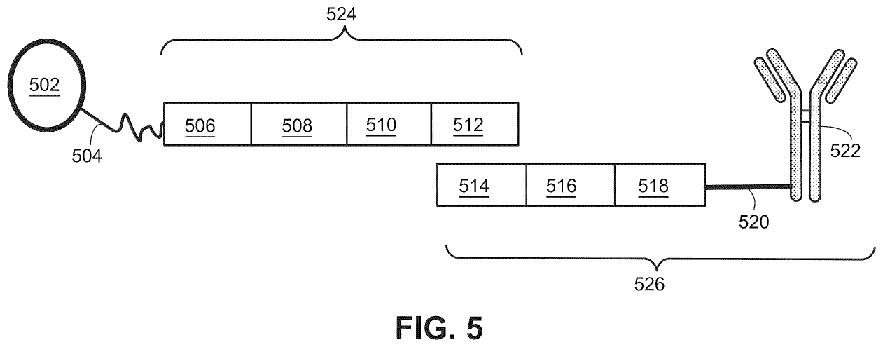

FIG. 5 is a schematic diagram depicting an exemplary interaction between a feature-immobilized capture probe 524 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain 516 to couple to the analyte binding moiety 522.

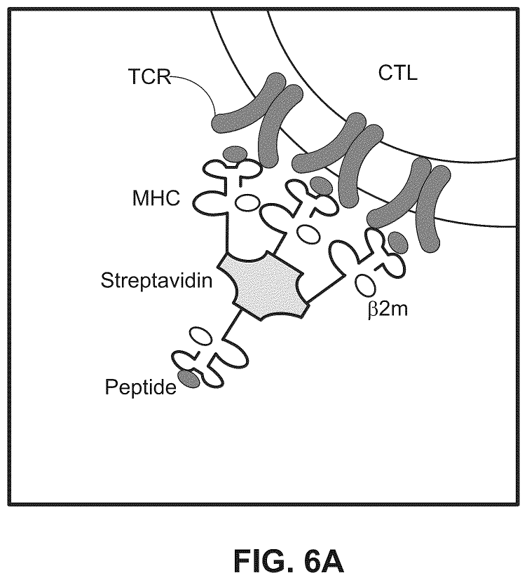

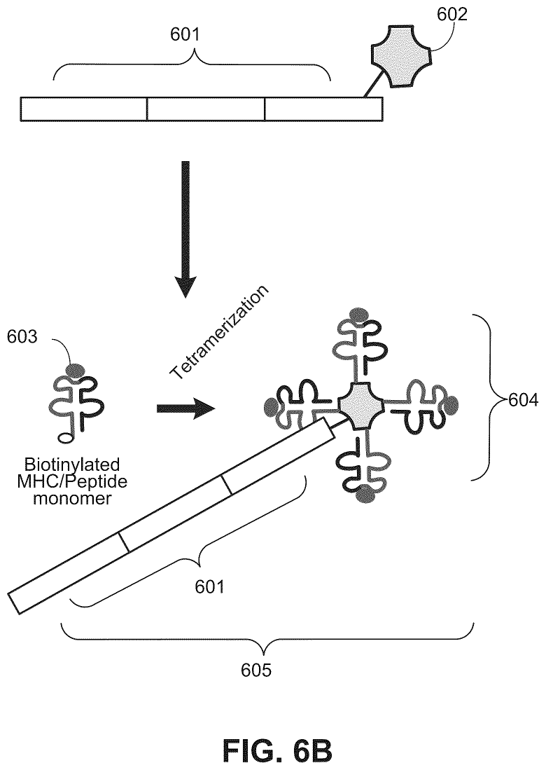

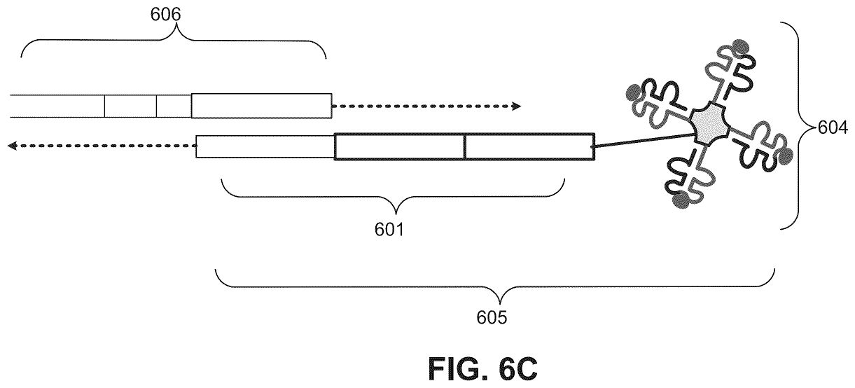

FIGS. 6A, 6B, and 6C are schematics illustrating how streptavidin cell tags can be utilized in an array-based system to produce a spatially-barcoded cell or cellular contents. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a TCR such that the streptavidin binds to a target T-cell via multiple MCH/TCR binding interactions. Multiple interactions synergize and can substantially improve binding affinity. Such improved affinity can improve labelling of T-cells and also reduce the likelihood that labels will dissociate from T-cell surfaces. As shown in FIG. 6B, a capture agent barcode domain 601 can be modified with streptavidin 602 and contacted with multiple molecules of biotinylated MHC 603 such that the biotinylated MHC 603 molecules are coupled with the streptavidin conjugated capture agent barcode domain 601. The result is a barcoded MHC multimer complex 605. As shown in FIG. 6B, the capture agent barcode domain sequence 601 can identify the MHC as its associated label and also includes optional functional sequences such as sequences for hybridization with other oligonucleotides. As shown in FIG. 6C, one example oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a gel bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of these corresponding sequences may be a complement of the original sequence in capture probe 606 or capture agent barcode domain 601. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from spatial barcode sequence on the capture agent barcode domain 601 may be used to identify the particular peptide MHC complex 604 bound on the surface of the cell (e.g., when using MHC-peptide libraries for screening immune cells or immune cell populations).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligation products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(c) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease (e.g., RNAse H). The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(c)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or scalable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

The sandwich process is described in PCT Patent Application Publication No. WO 2020/123320, which is incorporated by reference in its entirety.

### II. Spatial Analytical Methodology and Increasing Capture Efficiency

### (a) Introduction

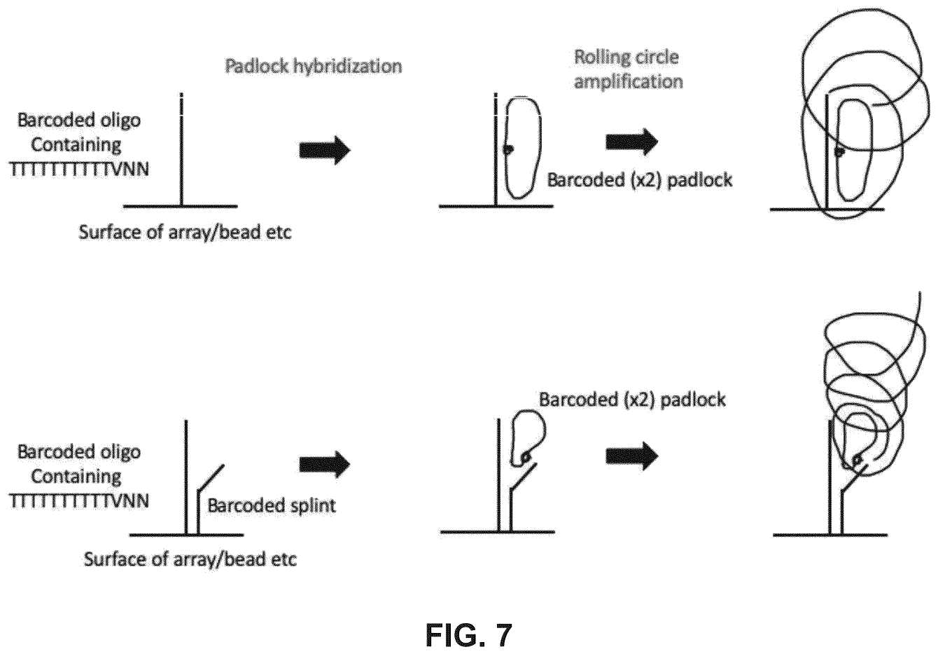

Provided herein are methods of increasing capture efficiency of a spatial array, e.g., a spatial array as described herein. In some embodiments, a method of increasing capture efficiency of a spatial array includes hybridizing a capture probe, e.g., any of the capture probes described herein, to an oligonucleotide. As disclosed herein, the capture probe comprises two sequences to which an oligonucleotide (i.e., a padlock probe) hybridizes. In some instances, in between the two sequences to which an oligonucleotide (i.e., a padlock probe) hybridizes is a capture domain sequence. In some instances, the oligonucleotide can be a padlock probe. A “padlock probe,” (also referred to as a padlock oligonucleotide, second probe, or second oligonucleotide) or as referred to herein, is an oligonucleotide that has, at its 5′ and 3′ ends, sequences that are complementary or nearly complementary to adjacent or nearby target sequences on a capture probe. Upon hybridization to the target sequences, the two ends of the padlock probe are either brought into contact or an end is extended until the two ends are brought into contact, allowing circularization of the padlock probe by ligation. In some embodiments, circularization of the padlock probe is performed using a ligation step. In some embodiments, a ligase is used. In some embodiments, the hybridized padlock probe is circularized using a polymerase (e.g., a DNA polymerase). See, for example, U.S. Pat. No. 7,358,047; Larsson et al. Nat Methods. 2004 December; 1(3):227-32; Larsson et al. Nat Methods. 2010 May; 7(5):395-7; and Ke et al. Nat Methods. 2013 September; 10(9):857-60; each of which is incorporated herein by reference in its entirety. After circularization of the padlock oligonucleotide, rolling circle amplification, for example, can be used to amplify the ligation product (see, for example, FIG. 7). Such methods can be useful for generating probes able to bind to more than one analyte (e.g., the probe can have more than one capture domain). Upon amplification, the sequence comprises multiple capture probe sequences to which analytes can bind. Thus, the methods disclosed herein provide the ability to capture a greater number of analytes in a given area of a biological sample compared to a capture probe comprising one capture domain.

Accordingly, a method of increasing capture efficiency of a spatial array can include contacting a substrate including a plurality of capture probes with a first oligonucleotide; wherein a capture probe of the plurality includes a first docking sequence, a second docking sequence, a barcode, and a capture domain; and the first oligonucleotide includes a first sequence complementary to the first docking sequence of the capture probe and a second sequence complementary to the second docking sequence of the capture probe; hybridizing the first docking sequence of the capture probe to the first complementary sequence of the first oligonucleotide and hybridizing the second docking sequence of the capture probe to the second complementary sequence of the first oligonucleotide; ligating the 3′ end of the first oligonucleotide to the 5′ end of the first oligonucleotide, thereby forming a circularized ligation product; and amplifying the ligation product using amplification (e.g., rolling circle amplification). In some embodiments, the plurality of capture probes are immobilized on the substrate.

In some embodiments, the methods disclosed herein include use of a splint oligonucleotide. In some embodiments, the splint oligonucleotide hybridizes to both the padlock probe and the capture probe on the array. See, for example, FIG. 7. In some embodiments, the second oligonucleotide includes sequences that are complementary (partially or fully) to a capture probe, a padlock probe, or both. In some embodiments, the second oligonucleotide facilitates hybridization of the first oligonucleotide to the capture probe. After hybridization, of the padlock probe to the capture probe and the splint oligonucleotide, ligation and amplification proceeds, thereby providing capture of a greater number of analytes in a given area of a biological sample compared to a capture probe comprising one capture domain.

Additional embodiments are provided herein.

### (b) Compositions for Increasing Capture Efficiency of a Spatial Array

### 1. Capture Probes and Arrays

Disclosed herein are capture probes that detect (e.g., hybridize to) analytes in a biological sample. In some instances, the capture probes comprise a plurality of nucleotides. In some instances, the capture probes comprise DNA.

In some instances, capture probes are placed on the substrate. In some instances, the capture probes are affixed to the surface of a substrate. In some instances, the capture probes are affixed to the surface of a substrate at the 5′ end of each capture probe. In some instances, the 3′ end of the capture probe is distal to the substrate.

The length of a capture probe as disclosed herein can vary. In some instances, the length of the capture probe is from 50 nucleotides to 500 nucleotides (e.g., about 50, 55, 60, 65, 70, 75, 80, 85, 90, 95, 100, 125, 150, 175, 200, 250, 300, 350, 400, 450, or 500 nucleotides). In some instances, the length of the capture probe is from 500 to 1000 nucleotides.

In some instances, the capture probe comprises a first docking sequence, a capture domain, and a second docking sequence. In some embodiments, the first docking sequence of the capture probe is within about 5 to about 200 nucleotides of the second docking sequence of the capture probe. For example, the first docking sequence of the capture probe is within about 5 to about 10, about 5 to about 20, about 5 to about 30, about 5 to about 40, about 5 to about 50, about 5 to about 60, about 5 to about 70, about 5 to about 80, about 5 to about 90, about 90 to about 100, about 80 to about 100, about 70 to about 100, about 60 to about 100, about 50 to about 200, about 40 to about 200, about 30 to about 200, about 20 to about 200, or about 10 to about 200 nucleotides to the second docking sequence of the capture probe.

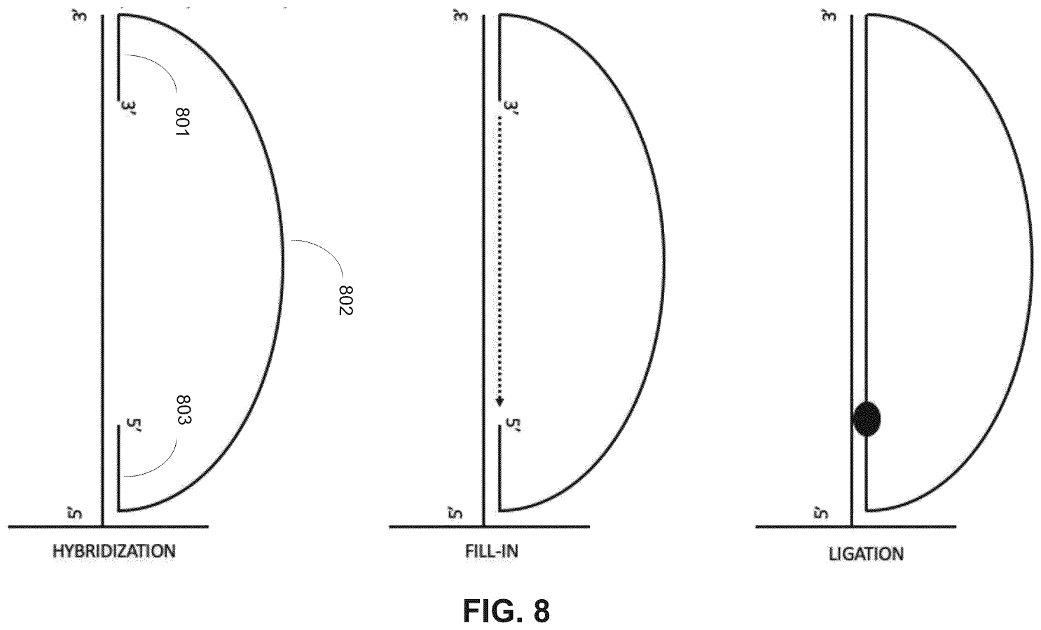

In some embodiments, after hybridization of the first oligonucleotide to the capture probe, there can be a gap between the first complementary sequence and the second complementary sequence (sec, for example, FIG. 8). For example, the capture probe can include one or more other domains between the first docking sequence and the second docking sequence. In instances in which the first docking sequence and the second docking sequence on the capture probe are not adjacent to one another, the capture probe include a capture domain between the two docking sequences. In some instances, the capture domain indiscriminately captures analytes of interest. For example, in some instances, the capture domain comprises a sequence that is complementary to a poly-adenylated sequence of an mRNA molecule. In some instances, the capture domain comprises a poly-thymine (i.e., poly(T)) sequence. In some instances, the poly(T) sequence is about 10 to about 100 nucleotides in length. It is appreciated that the sequence of the capture domain should be long enough in order to capture an analyte.

In some instance, the capture domain is designed to detect one or more specific analytes of interest. For example, a capture domain can be designed so that it comprises a sequence that is complementary or substantially complementary to one analyte of interest. Thus, the presence of a single analyte can be detected. Alternatively, the capture domain can be designed so that it comprises a sequence that is complementary or substantially complementary to a conserved region of multiple related analytes. In some instances, the multiple related analytes are analytes that function in the same or similar cellular pathways or that have conserved homology and/or function. The design of the capture probe can be determined based on the intent of the user and can be any sequence that can be used to detect an analyte of interest. In some embodiments, the capture domain sequence can therefore be random, semi-random, defined or combinations thereof, depending on the target analyte(s) of interest.

In some embodiments, the first docking sequence of the capture probe is adjacent to the second docking sequence of the capture probe. For example, in some embodiments, the first complementary sequence of the first oligonucleotide and the second complementary sequence of the first oligonucleotide hybridize to a portion of the capture probe such that the first complementary sequence of the first oligonucleotide and the second complementary sequence of the first oligonucleotide are within about 1, about 2, about 3, about 4, or about 5, about 6, about 7, about 8, about 9, about 10, about 15, about 20, about 30, about 40, about 50, about 60, about 70, about 80, about 90, about 100, about 125, about 150, about 175, about 200, about 250, about 300, about 350, about 400, about 450, about 500, or more nucleotides of each other on the capture probe. In instances in which the first docking sequence and the second docking sequence are adjacent to one another, the capture domain can be located on the padlock probe, discussed below.

In some instances, the capture probes includes one or more additional sequences. In some instances, the capture probe comprises a spatial barcode. In some instances, the spatial barcode 5′ to the first docking site. In some instances, the spatial barcode is 3′ to the second docking site. In some instances, the spatial barcode is between the first docking site and the second docking site on the capture probe. The spatial barcode is a unique sequence that provides the location of the capture probe (and ultimately the analyte in the biological sample).

In some instances, the capture probe comprises one or more functional domains. For instance, in some embodiments, the capture probe includes a unique molecular identifier. A unique molecular identifier is a contiguous nucleic acid segment or two or more non-contiguous nucleic acid segments that function as a label or identifier for a particular analyte, or for a capture probe that binds a particular analyte (e.g., via the capture domain). The UMI can include from about 6 to about 20 or more nucleotides within the sequence of the capture probes. In some embodiments, the length of a UMI sequence can be about 6, 7, 8, 9, 10, 11, 12, 13, 14, 15, 16, 17, 18, 19, 20 nucleotides or longer. In some embodiments, the length of a UMI sequence can be at least about 6, 7, 8, 9, 10, 11, 12, 13, 14, 15, 16, 17, 18, 19, 20 nucleotides or longer. In some embodiments, the length of a UMI sequence is at most about 6, 7, 8, 9, 10, 11, 12, 13, 14, 15, 16, 17, 18, 19, 20 nucleotides or shorter.

In some instances, the capture probe comprises a cleavage domain. The cleavage domain represents the portion of the probe that is used to reversibly attach the probe to an array feature, as will be described further herein. Further, one or more segments or regions of the capture probe can optionally be released from the array feature by cleavage of the cleavage domain. As an example, spatial barcodes and/or universal molecular identifiers (UMIs) can be released by cleavage of the cleavage domain.

In some embodiments, a cleavage domain is absent from the capture probe. Examples of substrates with attached capture probes lacking a cleavage domain are described for example in Macosko et al., (2015) Cell 161, 1202-1214, the entire contents of which are incorporated herein by reference.

In some embodiments, the region of the capture probe corresponding to the cleavage domain can be used for some other function. For example, an additional region for nucleic acid extension or amplification can be included where the cleavage domain would normally be positioned. In such embodiments, the region can supplement the functional domain or even exist as an additional functional domain. In some embodiments, the cleavage domain is present but its use is optional.

In some instances, the capture probe comprises any combination of any of the above-described sequences.

### 2. Padlock Probes

Also disclosed herein is a padlock probe (or padlock oligonucleotide, second probe, or second nucleotide). The padlock probe includes a plurality of nucleotides. In some instances, the padlock probe comprises DNA.

The length of a padlock probe as disclosed herein can vary. In some instances, the length of the padlock probe is from 50 nucleotides to 500 nucleotides (e.g., about 50, 55, 60, 65, 70, 75, 80, 85, 90, 95, 100, 125, 150, 175, 200, 250, 300, 350, 400, 450, or 500 nucleotides). In some instances, the length of the padlock probe is from 500 to 1000 nucleotides.

Referring to FIG. 8, the padlock probe comprises from 3′ to 5′, a sequence 801 complementary (or substantially complementary) to the first docking sequence, a padlock sequence 802, and a sequence 803 complementary (or substantially complementary) to the second docking sequence. In some instances, the sequence 801 is called the first docking padlock sequence. In some instances, the sequence 803 is called the second docking padlock sequence.

In some instances, the first docking padlock sequence comprises a sequence that is complementary to a portion of the first docking sequence of the capture probe. In some instances, the first docking padlock sequences is from 10 to 100 nucleotides in length. In some instances, the first docking padlock sequence is about 75%, 80%, 85%, 90%, 95%, or 100% complementary to the first docking sequence of the capture probe. The first docking padlock sequence can be designed using any sequence of interest so long as hybridization is achieved. In some instances, the first docking padlock sequence (and its complementary sequence) is not found in the genome of the organism of interest (e.g., not found in human, mouse, rat genome, etc.). One skilled in the art can design a sequence so that off-targets are not bound, or not substantially bound, to the first docking padlock sequence.

In some instances, the second docking padlock sequence comprises a sequence that is complementary to a portion of the second docking sequence of the capture probe. In some instances, the second docking padlock sequences is from 10 to 100 nucleotides in length. In some instances, the second docking padlock sequence is about 75%, 80%, 85%, 90%, 95%, or 100% complementary to the second docking sequence of the capture probe. The second docking padlock sequence can be designed using any sequence of interest so long as hybridization is achieved. In some instances, the second docking padlock sequence (and its complementary sequence) is not found in the genome of the organism of interest (e.g., not found in human, mouse, rat genome, etc.). One skilled in the art can design a sequence so that off-targets are not bound, or not substantially bound, to the second docking padlock sequence.

In some instances, the padlock sequence 802 is a DNA sequence. In some instances, the padlock sequence 802 is about 50 to 500 nucleotides in length (i.e., about 50, about 100, about 150, about 200, about 250, about 300, about 350, about 400, about 450, about 500). In some instances, the padlock sequence 802 can be designed to include one or more functional sequences. For instance, in some embodiments, the padlock sequence 802 comprises a capture domain sequence (e.g., a poly(T) sequence or a random, semi-random, or defined sequence as described above). In some instances, the capture domain sequence of the padlock is utilized when the first docking sequence and the second docking sequence are adjacent to one another on the capture probe. In some instances, the padlock sequence 802 comprises a primer sequence that can be used for amplification of the padlock probe. In some instances, the padlock sequence 802 comprises a cleaving domain as previously described.

In some embodiments, the padlock probe includes a functional sequence, e.g., any of the functional sequences described herein. In some embodiments, the padlock probe includes a 3′OH group. In some embodiments, the 3′OH group is a primer for the rolling circle amplification.

### 3. Splint Oligonucleotides

In some instances, a splint oligonucleotide is used to facilitate hybridization of the padlock probe to the capture probe. Referring to FIG. 7 (bottom panel), in some instances, the splint oligonucleotide comprises sequences that are complementary to the capture probe and to the padlock probe. In some instances, a portion of the splint oligonucleotide is complementary to a portion of the capture probe. In some instances, a portion of the splint oligonucleotide is complementary to a portion of the padlock probe. In some instances, the splint oligonucleotide comprises a sequence that is complementary to the first docking padlock sequence. In some instance, the splint oligonucleotide comprises a sequence that is complementary to the second docking padlock sequence.

In some embodiments, a portion of the splint oligonucleotide is at least 80%, at least 85%, at least 90%, at least 95%, at least 96%, at least 97%, at least 98% or at least 99% complementary to the capture probe. In some embodiments, a portion of the splint oligonucleotide is fully complementary to the capture probe.

In some embodiments, a portion of the splint oligonucleotide is at least 80%, at least 85%, at least 90%, at least 95%, at least 96%, at least 97%, at least 98% or at least 99% complementary to the first docking padlock sequence. In some embodiments, a portion of the splint oligonucleotide is fully complementary to the first docking padlock sequence.

In some embodiments, a portion of the splint oligonucleotide is at least 80%, at least 85%, at least 90%, at least 95%, at least 96%, at least 97%, at least 98% or at least 99% complementary to the second docking padlock sequence. In some embodiments, a portion of the splint oligonucleotide is fully complementary to the second docking padlock sequence.

The splint oligonucleotide includes a plurality of nucleotides. In some instances, the splint oligonucleotide comprises DNA.

The length of a splint oligonucleotide as disclosed herein can vary. In some instances, the length of the splint oligonucleotide is from 50 nucleotides to 500 nucleotides (e.g., about 50, 55, 60, 65, 70, 75, 80, 85, 90, 95, 100, 125, 150, 175, 200, 250, 300, 350, 400, 450, or 500 nucleotides). In some instances, the length of the splint oligonucleotide is from 500 to 1000 nucleotides.

### (c) Methods for Increasing Capture Efficiency of a Spatial Array

### 1. Probe Assembly, Padlock Hybridization, and Padlock Ligation

In some instances, probes are printed on a substrate. Arrays can be prepared by a variety of methods. In some embodiments, arrays are prepared through the synthesis (e.g., in situ synthesis) of oligonucleotides on the array, or by jet printing or lithography. For example, light-directed synthesis of high-density DNA oligonucleotides can be achieved by photolithography or solid-phase DNA synthesis. To implement photolithographic synthesis, synthetic linkers modified with photochemical protecting groups can be attached to a substrate and the photochemical protecting groups can be modified using a photolithographic mask (applied to specific areas of the substrate) and light, thereby producing an array having localized photo-deprotection. Many of these methods are known in the art, and are described e.g., in Miller et al., “Basic concepts of microarrays and potential applications in clinical microbiology.” Clinical Microbiology Reviews 22.4 (2009): 611-633; US201314111482A; U.S. Pat. No. 9,593,365B2; US2019203275; and WO2018091676, which are each incorporated herein by reference in its entirety. Additional disclosure of capture probe assembly is provided in WO 2020/176788 and/or U.S. Patent Application Publication No. 2020/0277663, each of which is incorporated by reference in its entirety.

In some embodiments, the capture probe and/or the splint oligonucleotide can be used to hybridize the padlock probe. For example, in some embodiments, a method of increasing capture efficiency of a spatial array includes contacting a substrate including a plurality of capture probes and a plurality of splint oligonucleotides with a padlock probe; wherein a capture probe of the plurality includes a first docking sequence, a spatial barcode, and a capture domain; a splint oligonucleotide includes a second docking sequence; the padlock probe includes a first complementary sequence that is complementary to the first docking sequence of the capture probe and a second complementary sequence that is complementary to the second docking sequence of the second oligonucleotide; hybridizing the first docking sequence of the capture probe to the first complementary docking sequence of the padlock probe and hybridizing the second docking sequence of the splint oligonucleotide to the second complementary docking sequence of the padlock probe; ligating the 3′ end of the first oligonucleotide to the 5′ end of the first oligonucleotide, thereby forming a ligation product; and amplifying the ligation product using rolling circle amplification to form an amplified ligation product. In some embodiments, the plurality of capture probes are immobilized on the substrate.

In some embodiments, the step of hybridizing the first docking sequence and the second docking sequence to the first docking padlock sequence and to the second docking padlock sequence is at a temperature from about 50° C. to about 75° C. For example, from about 50° C. to about 70° C., about 50° C. to about 65° C., about 50° C. to about 60° C., about 50° C. to about 55° C., about 70° C. to about 75° C., about 65° C. to about 75° C., about 60° C. to about 75° C., or about 55° C. to about 75° C. In some embodiments, the first temperature is from about 55° C. to about 70° C., or from about 60° C. to about 65° C.

In some instances, when a splint oligonucleotide is used, the splint oligonucleotide and the padlock probe are added to the array at the same time. In some instances, the splint oligonucleotide is added prior to addition of the padlock probe, allowing the splint oligonucleotide first to hybridize to the capture probe.

After hybridization, in some embodiments, the substrate can be treated with one or more enzymes and/or one or more reagents, as described herein. For example, referring to FIG. 8 (middle panel), one or more DNA polymerase enzymes and dNTPs can be used to fill in the gap between the first docking padlock sequence and the second docking padlock sequence (dotted line with arrow).

In some embodiments, a polymerase catalyzes synthesis of a complementary strand of the ligation product, creating a double-stranded ligation product. In some instances, the polymerase is DNA polymerase. In some embodiments, the polymerase has 5′ to 3′ polymerase activity. In some embodiments, the polymerase has 3′ to 5′ exonuclease activity for proofreading. In some embodiments, the polymerase has 5′ to 3′ polymerase activity and 3′ to 5′ exonuclease activity for proofreading.

In some instances, the methods further include ligating the two ends of the padlock probe. In some instances, the ligation is an enzymatic ligation reaction, using a ligase (e.g., T4 RNA ligase (Rn12), a SplintR ligase, a single stranded DNA ligase, or a T4 DNA ligase). See, e.g., Zhang et al.; RNA Biol. 2017; 14(1): 36-44, which is incorporated by reference in its entirety, for a description of KOD ligase. Following the enzymatic ligation reaction, the padlock probe may be considered ligated. In some embodiments, the enzymatic ligation utilizes one or more of T4 DNA ligase, Tth DNA ligase, Taq DNA ligase, Thermococcus sp. (strain 90N) DNA ligase, and Ampligase™. Derivatives, e.g., sequence-modified derivatives, and/or mutants thereof, can also be used.

In some embodiments, a padlock probe may comprise a reactive moiety such that, upon hybridization to the target and exposure to appropriate ligation conditions, the padlock probe may ligate to itself. In some embodiments, a padlock probe that includes a reactive moiety is ligated chemically. A reactive moiety of a probe may be selected from the non-limiting group consisting of azides, alkynes, nitrones (e.g., 1,3-nitrones), strained alkenes (e.g., trans-cycloalkenes such as cyclooctenes or oxanorbornadiene), tetrazines, tetrazoles, iodides, thioates (e.g., phorphorothioate), acids, amines, and phosphates. For example, the first reactive moiety of a padlock probe may comprise an azide moiety, and a second reactive moiety of a padlock probe may comprise an alkyne moiety. The first and second reactive moieties may react to form a linking moiety. A reaction between the first and second reactive moieties may be, for example, a cycloaddition reaction such as a strain-promoted azide-alkyne cycloaddition, a copper-catalyzed azide-alkyne cycloaddition, a strain-promoted alkyne-nitrone cycloaddition, a Diels-Alder reaction, a [3+2] cycloaddition, a [4+2] cycloaddition, or a [4+1] cycloaddition; a thiol-ene reaction; a nucleophilic substation reaction; or another reaction. In some cases, reaction between the first and second reactive moieties may yield a triazole moiety or an isoxazoline moiety. A reaction between the first and second reactive moieties may involve subjecting the reactive moieties to suitable conditions such as a suitable temperature, pH, or pressure and providing one or more reagents or catalysts for the reaction. For example, a reaction between the first and second reactive moieties may be catalyzed by a copper catalyst, a ruthenium catalyst, or a strained species such as a difluorooctyne, dibenzylcyclooctyne, or biarylazacyclooctynone. Reaction between a first reactive moiety of a first probe hybridized to a first target region (e.g., a first target sequence or first portion) of the nucleic acid molecule and a second reactive moiety of a third probe oligonucleotide hybridized to a second target region (e.g., a first target sequence or a first portion) of the nucleic acid molecule may link the first probe and the second probe to provide a ligated probe. Upon linking, the padlock probe may be considered ligated. Accordingly, reaction of the first and second reactive moieties may comprise a chemical ligation reaction such as a copper-catalyzed 5′ azide to 3′ alkyne “click” chemistry reaction to form a triazole linkage between the padlock probe sequences. In other non-limiting examples, an iodide moiety may be chemically ligated to a phosphorothioate moiety to form a phosphorothioate bond, an acid may be ligated to an amine to form an amide bond, and/or a phosphate and amine may be ligated to form a phosphoramidate bond.

In some embodiments, the methods described herein further includes a step of washing away any oligonucleotides (i.e., splint oligonucleotides or padlock probes) that do not hybridize to the capture domain. Steps of washing to remove unbound oligonucleotides can be performed using any of the wash methods described herein or known in the art (e.g., 1×SSC).

### 2. Padlock Probe Amplification

In some instances, after hybridizing the padlock probe to the capture probe, the padlock probe (i.e., the ligated padlock probe) is amplified. Amplifying the ligated padlock probe can include an isothermal or a non-isothermal amplification reaction. In some embodiments, amplifying of the ligation product comprises using rolling circle amplification. In some embodiments, amplification includes use of a polymerase as disclosed herein. In some embodiments, amplification includes use of a Phi29 polymerase. In some embodiments, a product of the rolling circle amplification results in the creation of multiple copies of a capture domain and multiple copies of a barcode.

In some embodiments, the ligated padlock probe comprises a functional sequence that is capable of binding to a primer used for amplification (referred to herein as the “amplification primer” or “primer used for amplification”). In some embodiments, the amplification primer is used to amplify the ligated padlock probe, thereby creating multiple copies of a capture domain.

### 3. Analyte or Analyte Derivative Hybridization and Spatial Analysis

After generating a plurality of padlock probes from the rolling circle amplification steps, in some instances, the biological sample is contacted with the substrate. The methods disclosed herein include detection of analytes that include protein and nucleic acids. In some instances, the method includes detection of nucleic acids. In some instances, the methods include detection of proteins.

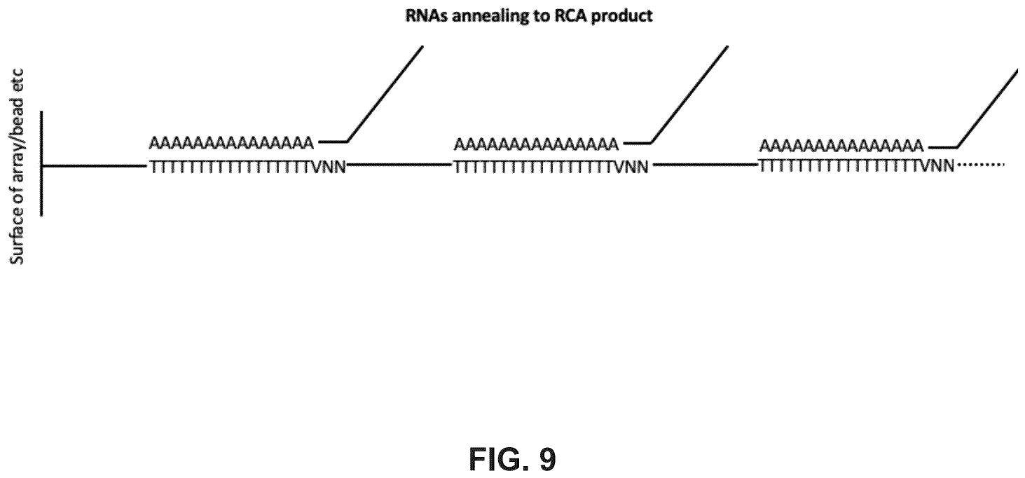

Referring to FIG. 9, after amplification of the padlock probe, the padlock probe comprises multiple copies of a capture domain. As shown in FIG. 9, multiple analytes, each of which comprise a sequence (e.g., a poly(A) sequence) complementary to the capture domain, hybridize to the amplified padlock probe. In some instances, the sequence complementary to the capture domain of the amplified padlock probe is a poly-adenylated (poly(A)) sequence. In some instances, the sequence complementary to the capture domain of the amplified padlock probe is designed to be specific to a sequence of interest (i.e., in a target analyte). In some instances, at least two, at least three, at least four, at least five, or more (e.g., 6, 7, 8, 9, 10, 15, 20 or more) analytes hybridize to a single amplified padlock probe.

In some instances, where multiple analytes hybridize to the same amplified padlock probe, the amplified padlock probe comprises a cleavage site so that each interaction between an analyte and the capture domain of an amplified padlock probe can be analyzed independently. In some instances, the cleavage site is located such that the spatial barcode is cleaved and migrates with the hybridized analyte/amplified padlock probe nucleic acid.

In the setting of nucleic acid detection, the nucleic acid analyte hybridizes directly to the capture probes on the amplified padlock probe. In the setting of protein detection, referring to FIG. 4, an analyte binding moiety 402 comprises a protein-binding moiety 404 and an oligonucleotide 408. In some instances, the analyte binding moieties are added to the biological sample. After association of the protein with the protein-binding moiety 404, the oligonucleotide is captured by a capture domain of the padlock probe. Thus, the analyte binding moiety as used herein can be considered an analyte derivate, and its oligonucleotide can be captured and analyzed in a similar way that a nucleic acid analyte can be analyzed. Embodiments for analyte binding moieties has been described previously e.g., in WO 2020/176788 and/or U.S. Patent Application Publication No. 2020/0277663, each of which is incorporated by reference in its entirety.

In some embodiments, the methods provided herein include a permeabilizing step in order to release the analytes from the biological sample. In some embodiments, permeabilization occurs using a protease. In some embodiments, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin. In some embodiments, after creating the padlock probe copies, the biological sample is permeabilized. In some embodiments, the biological sample is permeabilized contemporaneously with or prior to contacting the biological sample with a padlock probe.

In some embodiments, methods provided herein include permeabilization of the biological sample such that the capture probe can more easily bind to the padlock probe (i.e., compared to no permeabilization). In some embodiments, reverse transcription (RT) reagents can be added to permeabilized biological samples. Incubation with the RT reagents can produce spatially-barcoded full-length cDNA from the captured analytes (e.g., polyadenylated mRNA). Second strand reagents (e.g., second strand primers, enzymes) can be added to the biological sample on the substrate to initiate second strand synthesis.

In some instances, the permeabilization step includes application of a permeabilization buffer to the biological sample. In some instances, the permeabilization buffer includes a buffer (e.g., Tris pH 7.5), MgCl₂, sarkosyl detergent (e.g., sodium lauroyl sarcosinate) or other detergent, enzyme (e.g., proteinase K, pepsin, collagenase, etc.), and nuclease free water. In some instances, the permeabilization step is performed at 37° C. In some instances, the permeabilization step is performed for about 20 minutes to 2 hours (e.g., about 20 minutes, about 30 minutes, about 40 minutes, about 50 minutes, about 1 hour, about 1.5 hours, or about 2 hours). In some instances, the releasing step is performed for about 40 minutes.

After permeabilization, in some instances, the analytes are captured by the capture domains of the padlock probe. In some embodiments, such methods of increasing capture efficiency of a spatial array described herein includes contacting the spatial array with the biological sample and allowing the analyte to interact with the padlock probes. In some embodiments, the determining step includes amplifying all or part of the analyte bound to the padlock probes and amplifying all or part of the spatial barcode and the target analyte, or compliments thereof.

In some embodiments, the method includes amplifying all or part of the analyte using isothermal amplification or non-isothermal amplification. In some embodiments, the amplifying creates an amplifyed product that includes (i) all or part of a sequence of the analyte bound to the first capture domain and/or the second capture domain, or a complement thereof, and (ii) all or a part of the sequence of the first spatial barcode and/or the second spatial barcode, or a complement thereof. In some embodiments, the associating step also includes determining (i) all or part of the sequence of the first spatial barcode and (ii) all or part of the sequence of the second spatial barcode and using the determined sequence of (i) and (ii) to identify the location of the analyte in the spatial array. In some embodiments, the determining step includes sequencing. A non-limiting example of sequencing that can be used to determine the sequence of the analyte and/or spatial barcodes (e.g., first and/or second spatial barcode) is in situ sequencing. In some embodiments, in situ sequencing is performed via sequencing-by-synthesis (SBS), sequential fluorescence hybridization, sequencing by ligation, nucleic acid hybridization, or high-throughput digital sequencing techniques. In some embodiments the analyte is RNA or DNA. In some embodiments, the analyte is a protein.

More particularly, after an analyte (e.g., a first analyte, a second analyte, etc.) has hybridized or otherwise been associated with the padlock probe according to any of the methods described above in connection with the general spatial cell-based analytical methodology, the barcoded constructs that result from hybridization/association are analyzed.

In some embodiments, after contacting a biological sample with a substrate that includes capture probes, a removal step can optionally be performed to remove all or a portion of the biological sample from the substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, provided herein are methods for spatially detecting an analyte (e.g., detecting the location of an analyte, e.g., a biological analyte) from a biological sample (e.g., present in a biological sample), the method comprising: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes and padlock probes, wherein the padlock probe captures the biological analyte; and (d) analyzing the captured biological analyte, thereby spatially detecting the biological analyte; wherein the biological sample is fully or partially removed from the substrate.

In some embodiments, a biological sample is not removed from the substrate. For example, the biological sample is not removed from the substrate prior to releasing a capture probe (e.g., a padlock probe or a capture probe bound to an analyte) from the substrate. In some embodiments, such releasing comprises cleavage of the padlock probe from the substrate (e.g., via a cleavage domain). In some embodiments, such releasing does not comprise releasing the padlock probe from the substrate (e.g., a copy of the padlock probe bound to an analyte can be made and the copy can be released from the substrate, e.g., via denaturation). In some embodiments, the biological sample is not removed from the substrate prior to analysis of an analyte bound to a padlock probe after it is released from the substrate. In some embodiments, the biological sample remains on the substrate during removal of a padlock probe from the substrate and/or analysis of an analyte bound to the padlock probe after it is released from the substrate. In some embodiments, the biological sample remains on the substrate during removal (e.g., via denaturation) of a copy of the padlock probe (e.g., complement). In some embodiments, analysis of an analyte bound to a padlock probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a padlock probe (e.g., a padlock probe bound to an analyte) from the substrate and/or analyzing an analyte bound to a padlock probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a padlock probe from the substrate.

In some embodiments, the methods provided herein include spatially detecting an analyte (e.g., detecting the location of an analyte, e.g., a biological analyte) from a biological sample (e.g., present in a biological sample) that include: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes and padlock probes, wherein the padlock probe captures the biological analyte; and (d) analyzing the captured biological analyte, thereby spatially detecting the biological analyte; where the biological sample is not removed from the substrate.

In some embodiments, provided herein are methods for spatially detecting a biological analyte of interest from a biological sample that include: (a) staining and imaging a biological sample on a substrate; (b) providing a solution comprising a permeabilization reagent to the biological sample on the substrate; (c) contacting the biological sample with an array on a substrate, wherein the array comprises one or more capture probe pluralities thereby allowing the one or more pluralities of capture probes to capture the biological analyte of interest; and (d) analyzing the captured biological analyte, thereby spatially detecting the biological analyte of interest; where the biological sample is not removed from the substrate.

In some embodiments, the method further includes subjecting a region of interest in the biological sample to spatial transcriptomic analysis. In some embodiments, one or more of the padlock probes includes a capture domain. In some embodiments, one or more of the padlock probes comprises a unique molecular identifier (UMI). In some embodiments, one or more of the padlock probes comprises a cleavage domain. In some embodiments, the cleavage domain comprises a sequence recognized and cleaved by uracil-DNA glycosylase, apurinic/apyrimidinic (AP) endonuclease (APE1), uracil-specific excision reagent (USER), and/or an endonuclease VIII. In some embodiments, one or more padlock probes do not comprise a cleavage domain and is not cleaved from the array.

In some instances, the padlock probe can be cleaved, separating each interaction between an analyte and the padlock probe.

In some embodiments, the cleaved padlock probe can be extended (an “extended padlock probe,” e.g., as described herein). For example, extending a padlock probe can include generating cDNA from a captured (hybridized) RNA. This process involves synthesis of a complementary strand of the hybridized nucleic acid, e.g., generating cDNA based on the captured RNA template (the RNA hybridized to the capture domain of the padlock probe). Thus, in an initial step of extending a padlock probe, e.g., the cDNA generation, the captured (hybridized) nucleic acid, e.g., RNA, acts as a template for the extension, e.g., a reverse transcription step.

In some embodiments, the padlock probe is extended using reverse transcription. For example, reverse transcription includes synthesizing cDNA (complementary or copy DNA) from RNA, e.g., (messenger RNA), using a reverse transcriptase. In some embodiments, reverse transcription is performed while the tissue is still in place, generating an analyte library, where the analyte library includes the spatial barcodes from the adjacent padlock probes. In some embodiments, the padlock probe is extended using one or more DNA polymerases.

In some embodiments, a padlock probe includes a primer for producing the complementary strand of a nucleic acid hybridized to the padlock probe, e.g., a primer for DNA polymerase and/or reverse transcription. The nucleic acid, e.g., DNA and/or cDNA, molecules generated by the extension reaction incorporate the sequence of the padlock probe. The extension of the padlock probe, e.g., a DNA polymerase and/or reverse transcription reaction, can be performed using a variety of suitable enzymes and protocols.

In some embodiments, a full-length DNA (e.g., cDNA) molecule is generated. In some embodiments, a “full-length” DNA molecule refers to the whole of the captured nucleic acid molecule. However, if a nucleic acid (e.g., RNA) was partially degraded in the tissue sample, then the captured nucleic acid molecules will not be the same length as the initial RNA in the tissue sample. In some embodiments, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Lucigen, Middleton, WI). In some embodiments, template switching oligonucleotides are used to extend cDNA in order to generate a full-length cDNA (or as close to a full-length cDNA as possible). In some embodiments, a second strand synthesis helper probe (a partially double stranded DNA molecule capable of hybridizing to the 3′ end of the extended padlock probe), can be ligated to the 3′ end of the extended probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9° N) DNA ligase (9° N™ DNA ligase, New England Biolabs), Ampligase™ (available from Lucigen, Middleton, WI), and SplintR (available from New England Biolabs, Ipswich, MA). In some embodiments, a polynucleotide tail, e.g., a poly(A) tail, is incorporated at the 3′ end of the extended probe molecules. In some embodiments, the polynucleotide tail is incorporated using a terminal transferase active enzyme.

In some embodiments, double-stranded extended padlock probes are treated to remove any unextended padlock probes prior to amplification and/or analysis, e.g., sequence analysis. This can be achieved by a variety of methods, e.g., using an enzyme to degrade the unextended probes, such as an exonuclease enzyme, or purification columns.

In some embodiments, extended padlock probes are amplified to yield quantities that are sufficient for analysis, e.g., via DNA sequencing. In some embodiments, the first strand of the extended padlock probes (e.g., DNA and/or cDNA molecules) acts as a template for the amplification reaction (e.g., a polymerase chain reaction).

In some embodiments, the amplification reaction incorporates an affinity group onto the extended padlock probe (e.g., RNA-cDNA hybrid) using a primer including the affinity group. In some embodiments, the primer includes an affinity group and the extended padlock probes includes the affinity group. The affinity group can correspond to any of the affinity groups described previously.

In some embodiments, the extended padlock probes including the affinity group can be coupled to a substrate specific for the affinity group. In some embodiments, the substrate can include an antibody or antibody fragment. In some embodiments, the substrate includes avidin or streptavidin and the affinity group includes biotin. In some embodiments, the substrate includes maltose and the affinity group includes maltose-binding protein. In some embodiments, the substrate includes maltose-binding protein and the affinity group includes maltose. In some embodiments, amplifying the extended padlock probes can function to release the extended probes from the surface of the substrate, insofar as copies of the extended probes are not immobilized on the substrate.

In some embodiments, the extended padlock probe or complement or amplicon thereof is released. The step of releasing the extended padlock probe or complement or amplicon thereof from the surface of the substrate can be achieved in a number of ways. In some embodiments, an extended padlock probe or a complement thereof is released from the array by nucleic acid cleavage and/or by denaturation (e.g., by heating to denature a double-stranded molecule).

In some embodiments, the extended padlock probe or complement or amplicon thereof is released from the surface of the substrate (e.g., array) by physical means. For example, where the extended padlock probe is indirectly immobilized on the array substrate, e.g., via hybridization to a surface probe, it can be sufficient to disrupt the interaction between the extended padlock probe and the surface probe. Methods for disrupting the interaction between nucleic acid molecules include denaturing double stranded nucleic acid molecules are known in the art. A straightforward method for releasing the DNA molecules (i.e., of stripping the array of extended probes) is to use a solution that interferes with the hydrogen bonds of the double stranded molecules. In some embodiments, the extended padlock probe is released by an applying heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended padlock probe from the substrate.

In some embodiments, where the extended padlock probe includes a cleavage domain, the extended padlock probe is released from the surface of the substrate by cleavage. For example, the cleavage domain of the extended padlock probe can be cleaved by any of the methods described herein. In some embodiments, the extended padlock probe is released from the surface of the substrate, e.g., via cleavage of a cleavage domain in the extended padlock probe, prior to the step of amplifying the extended padlock probe.

In some embodiments, probes complementary to the extended padlock probe can be contacted with the substrate. In some embodiments, the biological sample can be in contact with the substrate when the probes are contacted with the substrate. In some embodiments, the biological sample can be removed from the substrate prior to contacting the substrate with probes. In some embodiments, the probes can be labeled with a detectable label (e.g., any of the detectable labels described herein). In some embodiments, probes that do not specially bind (e.g., hybridize) to an extended padlock probe can be washed away. In some embodiments, probes complementary to the extended padlock probe can be detected on the substrate (e.g., imaging, any of the detection methods described herein).

In some embodiments, probes complementary to an extended padlock probe can be about 4 nucleotides to about 100 nucleotides long. In some embodiments, probes (e.g., detectable probes) complementary to an extended padlock probe can be about 10 nucleotides to about 90 nucleotides long. In some embodiments, probes (e.g., detectable probes) complementary to an extended padlock probe can be about 20 nucleotides to about 80 nucleotides long. In some embodiments, probes (e.g., detectable probes) complementary to an extended padlock probe can be about 30 nucleotides to about 60 nucleotides long. In some embodiments, probes (e.g., detectable probes) complementary to an extended padlock probe can be about 40 nucleotides to about 50 nucleotides long. In some embodiments, probes (e.g., detectable probes) complementary to an extended padlock probe can be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 nucleotides long.

In some embodiments, about 1 to about 100 probes can be contacted to the substrate and specifically bind (e.g., hybridize) to an extended padlock probe. In some embodiments, about 1 to about 10 probes can be contacted to the substrate and specifically bind (e.g., hybridize) to an extended padlock probe. In some embodiments, about 10 to about 100 probes can be contacted to the substrate and specifically bind (e.g., hybridize) to an extended padlock probe. In some embodiments, about 20 to about 90 probes can be contacted to the substrate and specifically bind (e.g., hybridize) to an extended padlock probe. In some embodiments, about 30 to about 80 probes (e.g., detectable probes) can be contacted to the substrate and specifically bind (e.g., hybridize) to an extended padlock probe. In some embodiments, about 40 to about 70 probes can be contacted to the substrate and specifically bind (e.g., hybridize) to an extended padlock probe. In some embodiments, about 50 to about 60 probes can be contacted to the substrate and specifically bind (e.g., hybridize) to an extended padlock probe. In some embodiments, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 probes can be contacted to the substrate and specifically bind (e.g., hybridize) to an extended capture padlock.

In some embodiments, the probes can be complementary to a single analyte (e.g., a single gene). In some embodiments, the probes can be complementary to one or more analytes (e.g., analytes in a family of genes). In some embodiments, the probes (e.g., detectable probes) can be for a panel of genes associated with a disease (e.g., cancer, Alzheimer's disease, Parkinson's disease).

In some instances, the padlock probe can be amplified or copied, creating a plurality of cDNA molecules. In some embodiments, cDNA can be denatured from the padlock probe template and transferred (e.g., to a clean tube) for amplification, and/or library construction. The spatially-barcoded cDNA can be amplified via PCR prior to library construction. The cDNA can then be enzymatically fragmented and size-selected in order to optimize for cDNA amplicon size. P5 and P7 sequences directed to capturing the amplicons on a sequencing flowcell (e.g., Illumina sequencing instruments) can be appended to the amplicons, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites. A skilled artisan will understand that additional or alternative sequences used by other sequencing instruments or technologies are also equally applicable for use in the aforementioned methods as the current methods are not limited to any a particular sequencing platform.

In some embodiments, where a sample is barcoded directly via hybridization with padlock probes or analyte capture agents hybridized, bound, or associated with either the cell surface, or introduced into the cell, as described above, sequencing can be performed on the intact sample.

A wide variety of different sequencing methods can be used to analyze the barcoded analyte or moiety. In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, sequence by synthesis sequencing, nanopore sequencing, and Polony sequencing), ligation methods, and microarray methods.

In some embodiments, a capture domain as described herein (e.g., on a padlock probe) is blocked, thereby preventing an unwanted hybridization to the capture domain. In some embodiments, a blocking probe or chemical moiety is used to block or modify the free 3′ end of the padlock probe capture domain. In some embodiments, a blocking probe can be hybridized to the padlock probe capture domain of the second probe to mask the free 3′ end of the padlock probe capture domain. In some embodiments, a blocking probe can be a hairpin probe or partially double stranded probe. In some embodiments, the free 3′ end of the padlock probe capture domain of the second probe can be blocked by chemical modification, e.g., addition of an azidomethyl group as a chemically reversible capping moiety such that the capture probes do not include a free 3′ end. Blocking or modifying the padlock probe capture domain, particularly at the free 3′ end of the padlock probe capture domain, prior to contacting second probe with the substrate, prevents hybridization of the second probe to the capture domain (e.g., prevents the capture of a poly(A) of a padlock probe capture domain to a poly(T) capture domain). In some embodiments, a blocking probe can be referred to as a padlock probe capture domain blocking moiety.

### (d) Biological Sample, Analytes and Sample Preparation

### 1. Biological Samples and Analytes

Methods disclosed herein can be performed on any type of sample. In some embodiments, the sample is a fresh tissue sample. In some instances, the biological sample is a tissue, a tissue section, an organ, an organism, or a cell culture sample. In some instances, the biological sample is a formalin-fixed, paraffin-embedded (FFPE) sample, a frozen sample, or a fresh sample.

In some embodiments, the analyte includes one or more of RNA, DNA, a protein, a small molecule, and a metabolite. In some embodiments, the analyte (e.g., target analyte) is a single-stranded oligonucleotide. In some embodiments, the single-stranded oligonucleotide is RNA. In some embodiments, the RNA is mRNA. In some embodiments, the mRNA is an mRNA of interest. In some embodiments, the multiple target analytes are detected. The multiple targets can, in some instances, include sequences that have at least 80%, at least 85%, at least 90%, at least 91%, at least 92%, at least 93%, at least 94%, at least 95%, at least 96%, at least 97%, at least 98%, or at least 99% sequence identity to each other. In some instances, the multiple targets each include one or more conserved sequences. In some instances, the multiple targets are mRNAs that encode for proteins that have a similar function. In some instances, the multiple targets are mRNAs that encode for proteins that function in the same or a similar cellular pathway.

Subjects from which biological samples can be obtained can be healthy or asymptomatic individuals that have or are suspected of having a disease (e.g., cancer) or a pre-disposition to a disease, and/or individuals that are in need of therapy or suspected of needing therapy. In some instances, the biological sample can include one or more diseased cells. A diseased cell can have altered metabolic properties, gene expression, protein expression, and/or morphologic features. Examples of diseases include inflammatory disorders, metabolic disorders, nervous system disorders, and cancer. In some instances, the biological sample includes cancer or tumor cells. Cancer cells can be derived from solid tumors, hematological malignancies, cell lines, or obtained as circulating tumor cells. In some instances, the biological sample is a heterogenous sample. In some instances, the biological sample is a heterogenous sample that includes tumor or cancer cells and/or stromal cells,

In some embodiments, the biological sample is from a human subject.

FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of poly(T) capture and reverse transcription of mRNA). In certain embodiments, methods provided herein enable sensitive measurement of specific genes of interest that otherwise might be missed with a whole transcriptomic approach.

In some instances, FFPE samples are stained (e.g., using H&E, immunofluorescence, etc.). The methods disclosed herein are compatible with staining methods that will allow for morphological context overlaid with transcriptomic analysis. However, depending on the need some samples may be stained with only a nuclear stain, such as staining a sample with only hematoxylin and not eosin, the use of DAPI, etc. when location of a cell nucleus is needed.

In some embodiments, a biological sample (e.g. tissue section) can be fixed with methanol, stained with hematoxylin and eosin, and imaged. In some embodiments, fixing, staining, and imaging occurs before one or more probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the substrate.

In some embodiments, the FFPE sample is deparaffinized, permeabilized, equilibrated, and blocked before target probe oligonucleotides are added. In some embodiments, deparaffinization includes the use of xylenes. In some embodiments, deparaffinization includes multiple washes with xylenes. In some embodiments, deparaffinization includes multiple washes with xylenes followed by removal of xylenes using multiple rounds of graded alcohol washes followed by washing the sample with water. In some aspects, the water is deionized water. In some embodiments, equilibrating and blocking includes incubating the sample in a pre-Hyb buffer. In some embodiments, the pre-Hyb buffer includes yeast tRNA. In some embodiments, permeabilizing a sample includes washing the sample with a phosphate buffer. In some embodiments, the buffer is PBS. In some embodiments, the buffer is PBST.

### 2. Imaging and Staining

In some instances, biological samples can be stained using a wide variety of stains and staining techniques. In some instances, the biological sample is a section of a tissue (e.g., a 10 μm section). In some instances, the biological sample is dried after placement onto a glass slide. In some instances, the biological sample is dried at 42° C. In some instances, drying occurs for about 1 hour, about 2, hours, about 3 hours, or until the sections become transparent. In some instances, the biological sample can be dried overnight (e.g., in a desiccator at room temperature).

In some embodiments, a sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the methods disclosed herein include imaging the biological sample. In some instances, imaging the sample occurs prior to deaminating the biological sample. In some instances, the sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some instances, the stain is an H&E stain.

In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some embodiments, biological samples can be destained. Methods of destaining or decoloring a biological sample are known in the art, and generally depend on the nature of the stain(s) applied to the sample. For example, H&E staining can be destained by washing the sample in HCl, or any other acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in an acid (e.g., HCl). In some embodiments, destaining can include adding HCl to a downstream solution (e.g., permeabilization solution). In some embodiments, destaining can include dissolving an enzyme used in the disclosed methods (e.g., pepsin) in an acid (e.g., HCl) solution. In some embodiments, after destaining hematoxylin with an acid, other reagents can be added to the destaining solution to raise the pH for use in other applications. For example, SDS can be added to an acid destaining solution in order to raise the pH as compared to the acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, immunofluorescence or immunohistochemistry protocols (direct and indirect staining techniques) can be performed as a part of, or in addition to, the exemplary spatial workflows presented herein. For example, tissue sections can be fixed according to methods described herein. The biological sample can be transferred to an array (e.g., capture probe array), wherein analytes (e.g., proteins) are probed using immunofluorescence protocols. For example, the sample can be rehydrated, blocked, and permeabilized (3×SSC, 2% BSA, 0.1% Triton X, 1 U/μl RNAse inhibitor for 10 minutes at 4° C.) before being stained with fluorescent primary antibodies (1:100 in 3×SSC, 2% BSA, 0.1% Triton X, 1 U/μl RNAse inhibitor for 30 minutes at 4° C.). The biological sample can be washed, coverslipped (in glycerol+1 U/μl RNAse inhibitor), imaged (e.g., using a confocal microscope or other apparatus capable of fluorescent detection), washed, and processed according to analyte capture or spatial workflows described herein.

In some instances, a glycerol solution and a cover slip can be added to the sample. In some instances, the glycerol solution can include a counterstain (e.g., DAPI).

As used herein, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some embodiments, optimizing permeabilization can be useful for identifying intracellular analytes. Permeabilization optimization can include selection of permeabilization agents, concentration of permeabilization agents, and permeabilization duration. Tissue permeabilization is discussed elsewhere herein.

In some embodiments, blocking an array and/or a biological sample in preparation of labeling the biological sample decreases nonspecific binding of the antibodies to the array and/or biological sample (decreases background). Some embodiments provide for blocking buffers/blocking solutions that can be applied before and/or during application of the label, wherein the blocking buffer can include a blocking agent, and optionally a surfactant and/or a salt solution. In some embodiments, a blocking agent can be bovine serum albumin (BSA), serum, gelatin (e.g., fish gelatin), milk (e.g., non-fat dry milk), casein, polyethylene glycol (PEG), polyvinyl alcohol (PVA), or polyvinylpyrrolidone (PVP), biotin blocking reagent, a peroxidase blocking reagent, levamisole, Carnoy's solution, glycine, lysine, sodium borohydride, pontamine sky blue, Sudan Black, trypan blue, FITC blocking agent, and/or acetic acid. The blocking buffer/blocking solution can be applied to the array and/or biological sample prior to and/or during labeling (e.g., application of fluorophore-conjugated antibodies) to the biological sample.

### 3. Preparation of Sample for Application of Probes

In some instances, the biological sample is deparaffinized. Deparaffinization can be achieved using any method known in the art. For example, in some instances, the biological samples is treated with a series of washes that include xylene and various concentrations of ethanol. In some instances, methods of deparaffinization include treatment with xylene (e.g., three washes at 5 minutes each). In some instances, the methods further include treatment with ethanol (e.g., 100% ethanol, two washes 10 minutes each; 95% ethanol, two washes 10 minutes each; 70% ethanol, two washes 10 minutes each; 50% ethanol, two washes 10 minutes each). In some instances, after ethanol washes, the biological sample can be washed with deionized water (e.g., two washes for 5 minutes each). It is appreciated that one skilled in the art can adjust these methods to optimize deparaffinization.

In some instances, the biological sample is decrosslinked. In some instances, the biological sample is decrosslinked in a solution containing TE buffer (comprising Tris and EDTA). In some instances, the TE buffer is basic (e.g., at a pH of about 9). In some instances, decrosslinking occurs at about 50° C. to about 80° C. In some instances, decrosslinking occurs at about 70° C. In some instances, decrosslinking occurs for about 1 hour at 70° ° C. Just prior to decrosslinking, the biological sample can be treated with an acid (e.g., 0.1M HCl for about 1 minute). After the decrosslinking step, the biological sample can be washed (e.g., with 1×PBST).

In some instances, the methods of preparing a biological sample for probe application include permeabilizing the sample. In some instances, the biological sample is permeabilized using a phosphate buffer. In some instances, the phosphate buffer is PBS (e.g., 1×PBS). In some instances, the phosphate buffer is PBST (e.g., 1×PBST). In some instances, the permeabilization step is performed multiple times (e.g., 3 times at 5 minutes each).

In some instances, the methods of preparing a biological sample for probe application include steps of equilibrating and blocking the biological sample. In some instances, equilibrating is performed using a pre-hybridization (pre-Hyb) buffer. In some instances, the pre-Hyb buffer is RNase-free. In some instances, the pre-Hyb buffer contains no bovine serum albumin (BSA), solutions like Denhardt's, or other potentially nuclease-contaminated biological materials.

In some instances, the equilibrating step is performed multiple times (e.g., 2 times at 5 minutes each; 3 times at 5 minutes each). In some instances, the biological sample is blocked with a blocking buffer. In some instances, the blocking buffer includes a carrier such as tRNA, for example yeast tRNA such as from brewer's yeast (e.g., at a final concentration of 10-20 μg/mL). In some instances, blocking can be performed for 5, 10, 15, 20, 25, or 30 minutes.

Any of the foregoing steps can be optimized for performance. For example, one can vary the temperature. In some instances, the pre-hybridization methods are performed at room temperature. In some instances, the pre-hybridization methods are performed at 4° C. (in some instances, varying the timeframes provided herein).

### (e) Kits

In some embodiments, also provided herein are kits that include one or more reagents to detect one or more analytes described herein. In some instances, the kit includes a substrate comprising a plurality of capture probes comprising a capture domain. In some instances, the kit includes a plurality of probes (e.g., capture probes, padlock probes, and splint oligonucleotides) as described herein.

A non-limiting example of a kit used to perform any of the methods described herein includes: (a) a substrate comprising a plurality of capture probes comprising a spatial barcode and a capture domain; (b) a system comprising padlock probes and splint oligonucleotides; and (c) instructions for performing the methods provided herein.

Another non-limiting example of a kit used to perform any of the methods described herein includes: (a) an array comprising a plurality of capture probes; (b) padlock probes and splint oligonucleotides; (c) a plurality of enzymes, including a polymerase and a ligase; and (d) instructions for performing the methods provided herein.

### EXAMPLES

### Example 1. Method for Increasing Capture Efficiency of a Spatial Array Using a Padlock Probe

A substrate with a plurality of capture probes is contacted with a first oligonucleotide. A capture probe of the plurality has a first docking sequence, a second docking sequence, a barcode, and a capture domain. The first oligonucleotide has a first complementary sequence complementary to the first docking sequence of the capture probe and a second complementary sequence complementary to the second docking sequence of the capture probe. The first docking sequence of the capture probe is hybridized to the first complementary sequence of the first oligonucleotide and the second docking sequence of the capture probe is hybridized to the second complementary sequence of the first oligonucleotide. The 3′ end of the first oligonucleotide is ligated to the 5′ end of the first oligonucleotide, thereby forming a ligation product; and the ligation product is amplified using rolling circle amplification.

### Example 2. Method for Increasing Capture Efficiency of a Spatial Array Using a Padlock Probe and a Splint Oligonucleotide

A substrate with a plurality of capture probes is contacted with a first oligonucleotide and a second oligonucleotide. A capture probe of the plurality has a first docking sequence, a barcode, and a capture domain. The second oligonucleotide has a second docking sequence, and the first oligonucleotide includes a first complementary sequence that is complementary to the first docking sequence of the capture probe and a second complementary sequence that is complementary to the second docking sequence of the second oligonucleotide. The first docking sequence of the capture probe is hybridized to the first complementary docking sequence of the first oligonucleotide, and the second docking sequence of the second oligonucleotide is hybridized to the second complementary docking sequence of the first oligonucleotide. The 3′ end of the first oligonucleotide is ligated to the 5′ end of the first oligonucleotide, thereby forming a ligation product. The ligation product is amplified using rolling circle amplification to form an amplified ligation product.

### Example 3. Method for Increasing Capture Efficiency of a Spatial Array Using an Extended Padlock Probe

A substrate having a plurality of capture probes is contacted with a first oligonucleotide. A capture probe of the plurality has a first docking sequence, a second docking sequence, a barcode, and a capture domain. The first oligonucleotide has a first complementary sequence complementary to the first docking sequence of the capture probe, and a second complementary sequence complementary to the first docking sequence of the capture probe. The first docking sequence and the second docking sequence of the capture probe are hybridized to the first complementary docking sequence and the second complementary docking sequence of the oligonucleotide. The first oligonucleotide is extended. The extended 3′ end of the first oligonucleotide is ligated to the 5′ end of the first oligonucleotide to form a ligation product; and the ligation product is amplified using rolling circle amplification to form an amplified ligation product.

What is claimed is:

1. A method for preparing a spatial array, the method comprising:
(a) contacting a substrate comprising a plurality of capture probes with a padlock probe, wherein:
(i) a capture probe of the plurality of capture probes comprises a first docking sequence, a spatial barcode, a capture domain, and a second docking sequence; and
(ii) the padlock probe comprises:
a first docking padlock sequence at its 3′ end that is complementary to the first docking sequence of the capture probe, and
a second docking padlock sequence at its 5′ end that is complementary to the second docking sequence of the capture probe;
(b) hybridizing the first docking sequence to the first docking padlock sequence and hybridizing the second docking sequence to the second docking padlock sequence;
(c) ligating the padlock probe, thereby generating a ligated padlock probe that comprises the capture domain or a complement thereof; and
(d) amplifying the ligated padlock probe, thereby generating an amplified padlock probe sequence with multiple copies of the capture domain.
